(19) United States
(12) Patent Application Publication
(10) Pub. No.: US 2026/0218108 A1
(43) Pub. Date: Jul. 30, 2026

(54) 3D MICROFLUIDIC TRANSFECTION DEVICES AND METHOD OF TRANSFECTING TARGET CELLS WITH A TRANSFECTION AGENT

(71) Applicant: University of Kentucky Research Foundation, Lexington, KY (US)

(72) Inventors: Guigen Zhang, Lexington, KY (US); Sheng Tong, Lexington, KY (US)

(21) Appl. No.: 19/145,282

(22) PCT Filed: Jan. 5, 2024
(86) PCT No.: PCT/US2024/010575

§ 371 Date: Jul. 2, 2025

Related U.S. Application Data
(60) Provisional application No. 63/478,713, filed on Jan. 6, 2023.

(51) Int. Cl. C12M 1/42; C12M 3/06; C12N 15/87

(52) U.S. Cl. CPC C12M 35/02; C12M 23/16; C12N 15/87

(57) ABSTRACT

A microfluidic transfection device, adapted for transfecting target cells with a transfection agent, includes a microfluidic flow channel and an electric field generator. The microfluidic device includes a flow channel for the target cells and the transfection agent suspended in a carrying solution, the flow channel having (a) an inlet, (b) bifurcation, and (c) an outlet. The electric field generator is adapted to generate an electric field across a width of the flow channel whereby the target cells continuously flowing through the flow channel are transfected with the transfection agent under combined physical perturbations including electrical field and hydro-pressure.

### RELATED APPLICATION

This application claims the benefit of U.S. Provisional Patent Application Ser. No. 63/478,713, filed on Jan. 6, 2023, the full disclosure of which is hereby incorporated by reference in its entirety.

### TECHNICAL FIELD

This document relates generally to 3D microfluidic devices and related methods for enabling transfection of cells in a continuous flow-through and clog-free manner with maximal uniformity, consistency and efficiency, and minimal adverse effects.

### BACKGROUND

As cell therapy becomes a part of modern medicine to treat cancer, technologies that can provide high efficiency, consistency and uniformity in cell transfection are urgently needed. Currently, ex vivo cell transfection is mainly done using cuvette-based instruments, with few flow-through systems to help speed up the transfection processes. Some of the challenges of current cell transfection technologies include varying efficiency due to inconsistent and nonuniform exposure of the cells to the electric field, batch-to-batch variations, and adverse effects on cells due to the high voltage (from 100s V to 1000s V) applied. New technologies are in urgent need to offer high-throughput, maximal uniformity and consistency in cell transfection, and minimal adverse effect on cells. These important capabilities will not only enable cell-therapy industry to meet the scale-up demands and stringent Good Manufacturing Practice (GMP) requirements, but also help lower the cost for cancer treatments to benefit patients.

The 3D microfluidic transfection device and related method technology disclosed in this document will provide exactly these important capabilities and more. In the new and improved microfluidic device, cells will be transfected in a continuous flow-through and clog-free manner. Prior to electric-field exposure, all cells, while moving along with the flow, will be forced to take a fixed path through the electric field and have their membranes aligned perpendicular to the electric field. In this way, all cells will be exposed to the electric field uniformly and consistently in a maximal and controllable amount.

### SUMMARY

In accordance with the purposes and benefits set forth herein, a new and improved 3D microfluidic device adapted for transfecting target cells with a transfection agent is provided. That microfluidic transfection device comprises, consists of or consists essentially of; (1) a flow channel for the target cells and the transfection agent suspended in a carrying solution, the flow channel having (a) an inlet, (b) a bifurcation (e.g. in the form of a vertically bifurcated channel segment) and (c) an outlet, and (2) an electric field generator adapted to generate an electric field across a width of the flow channel whereby the target cells continuously flowing through the flow channel are transfected with the transfection agent via combined physical perturbations including electrical field and hydro pressure to ensure better cell health.

In at least some of the many possible embodiments of the 3D microfluidic transfection device, the electric field generator includes (a) electrodes positioned adjacent to the flow channel and (b) a voltage source connected to the electrodes and adapted for applying a voltage potential across the electrodes. The electrodes may include a first set of electrodes for electroporation on substantially opposite sides of the flow channel. The vertical bifurcation in the flow channel may be provided downstream from the first set of electrodes to introduce a change in hydro pressure to cells in the flow channel to facilitate enhanced delivery of transfection agent and at the same time provide additional tuning of physical perturbations to ensure better cell health. The electric field generator may further include a second set of electrodes on substantially opposite sides of the bifurcation.

In one particularly useful embodiment, the first set of electrodes include first longitudinal axes substantially perpendicular to the flow channel and in a common plane with the flow channel. The second set of electrodes include second longitudinal axes perpendicular to and offset from the flow channel at the bifurcation.

In at least some embodiments, the vertical bifurcation merges back again into a single flow channel downstream from the second set of electrodes and upstream from the outlet.

The flow channel may have a rectangular cross section having a height H and a width W, where H>W. Turns of the intermediate arcuate section and the width W may share a common plane. The flow channel may have a width of between about 10 μm and about 250 μm and an aspect ratio of channel cross sectional area (H/W) of from about 1.5 to about 4.5.

In at least some embodiments of the 3D microfluidic transfection device, the flow channel includes an intermediate arcuate section having at least one turn. In other possible embodiments, the flow channel includes an intermediate arcuate section in the form of a spiral including about 1 to about 10 complete loops. The intermediate arcuate section may include an innermost loop having a radius of between about 0.5 mm to about 25 mm and the outermost loop having a radius of between about 0.525 mm and about 30 mm.

In accordance with another aspect, a microfluidic transfection device, comprises, consists of or consists essentially of: (1) a flow channel for the target cells and the transfection agent suspended in a carrying solution, the flow channel having (a) an inlet, (b) an intermediate arcuate section adapted to advantageously position target cells within the flow channel for transfection, (c) an outlet section including a bifurcation, and (d) an outlet, and (2) an electric field generator adapted to generate an electric field across a width of the flow channel adjacent the bifurcation whereby the target cells continuously flowing through the flow channel are transfected with the transfection agent.

In accordance with an additional aspect, a method of transfecting target cells with a transfection agent in a suspension, comprises, consists of or consists essentially of: (1) introducing the suspension into an inlet of a flow channel, (2) flowing the suspension through the flow channel from the inlet serially through a bifurcation to an outlet of the flow channel, and (3) applying an electric field across a width of the flow channel adjacent the bifurcation whereby the target cells continuously flowing through the flow channel are transfected with the transfection agent under combined physical perturbations including electrical field and hydro-pressure.

The method may include applying a voltage potential across a first set of electrodes on substantially opposite sides of the outlet section of the flow channel. The method may include passing the suspension with the target cells and the transfection agent through the bifurcation downstream from the first set of electrodes. The method may include applying a voltage potential across a second set of electrodes on substantially opposite sides of the bifurcation in the flow channel.

The method may include passing the suspension with the target cells and the transfection agent through the flow channel of rectangular cross section having a height H and a width W, where H>W, and wherein turns of the intermediate arcuate section and the width W share a common plane. In at least some embodiments, the method includes at least one turn in an intermediate arcuate section of the flow channel. In other embodiments, the method includes a spiral having about 1 to about 10 complete loops in an intermediate arcuate section of the flow channel.

In the following description, there are shown and described several different embodiments of the new and improved 3D microfluidic transfection device and method for enabling transfection of cells in a continuous flow-through and clog-free manner with maximal uniformity, consistency and efficiency, and minimal adverse effects. As it should be realized, the fluidic transfection device and method are capable of other, different embodiments and their several details are capable of modification in various, obvious aspects all without departing from the device and method as set forth and described in the following claims. Accordingly, the descriptions should be regarded as illustrative in nature and not as restrictive.

### BRIEF DESCRIPTION OF THE DRAWING FIGURES

The accompanying drawing figures incorporated herein and forming a part of the specification, illustrate certain aspects of the device and method and together with the description serve to explain certain principles thereof. A person of ordinary skill in the art will readily recognize from the following discussion that alternative embodiments of the device and method may be employed without departing from the principles described below.

FIG. 1A is a perspective view of a first possible embodiment of the microfluidic transfection device wherein the intermediate arcuate section includes a spiral of about three complete loops.

FIG. 1B illustrates how cells in solution are passed through the flow channel of the microfluidic transfection device at the Point A illustrated in FIG. 1A.

FIG. 1C illustrates how cells in solution are passed through the flow channel of the microfluidic transfection device at the Point B illustrated in FIG. 1A.

FIG. 2 illustrates another possible embodiment of the microfluidic transfection device wherein the intermediate arcuate section includes two 180-degree turns.

FIG. 3A illustrates yet another possible embodiment of the microfluidic transfection device wherein the intermediate arcuate section includes four 180-degree turns and the electrical field generator includes pairs of horizontal electrodes and vertical electrodes.

FIG. 3B illustrates a sample electric field generated by a pair of horizontal electrodes.

FIG. 3C illustrates sample pulse-like electric fields when five pair of vertical electrodes of different diameter are used.

FIG. 4 is a graph illustrating hydro-pressure forces as a function of aspect ratio of the cross section of the flow channel.

FIG. 5 illustrates the microfluidic transfection device of FIG. 3A held in a housing.

Reference will now be made in detail to the present preferred embodiments of the fluidic transfection device and method.

### DETAILED DESCRIPTION

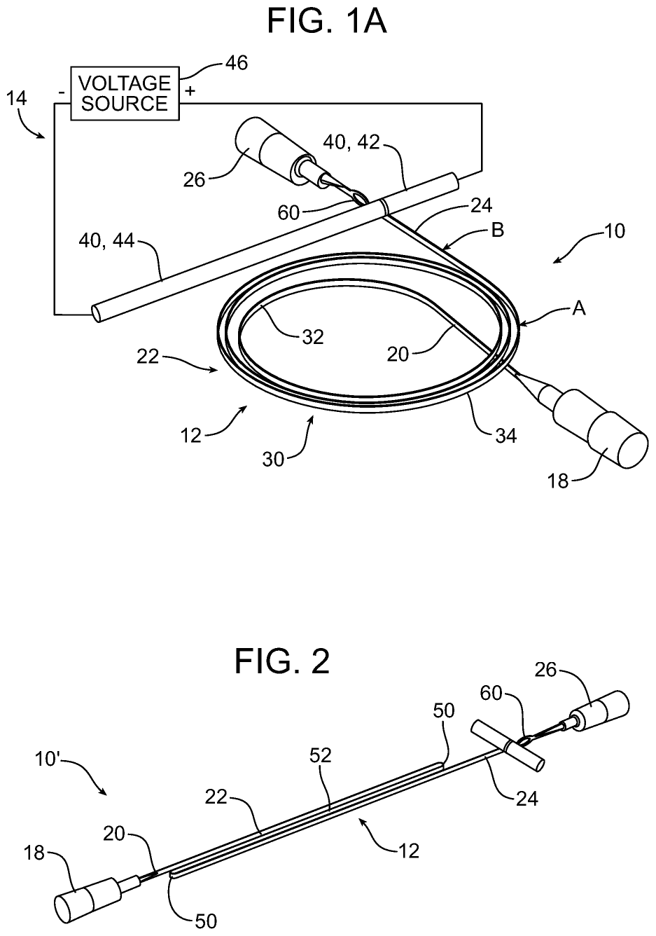

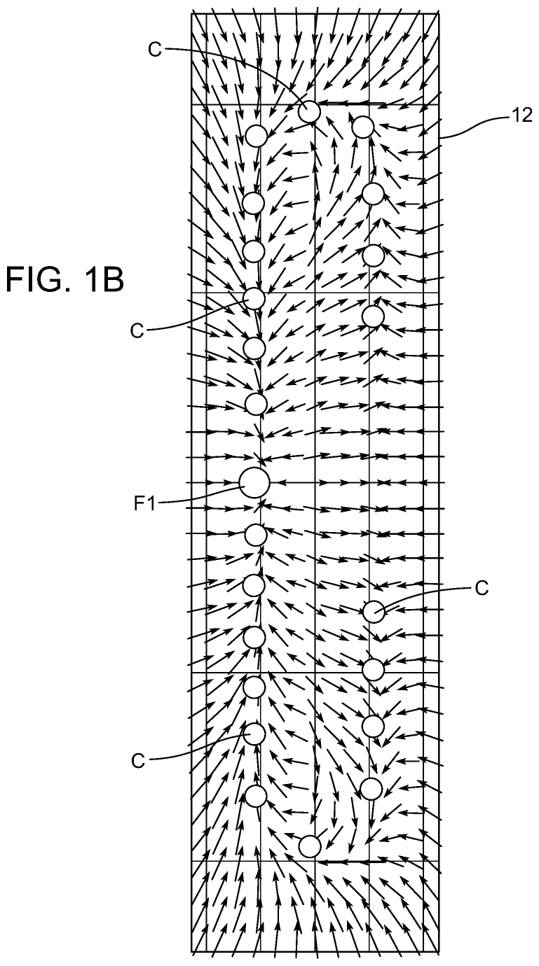

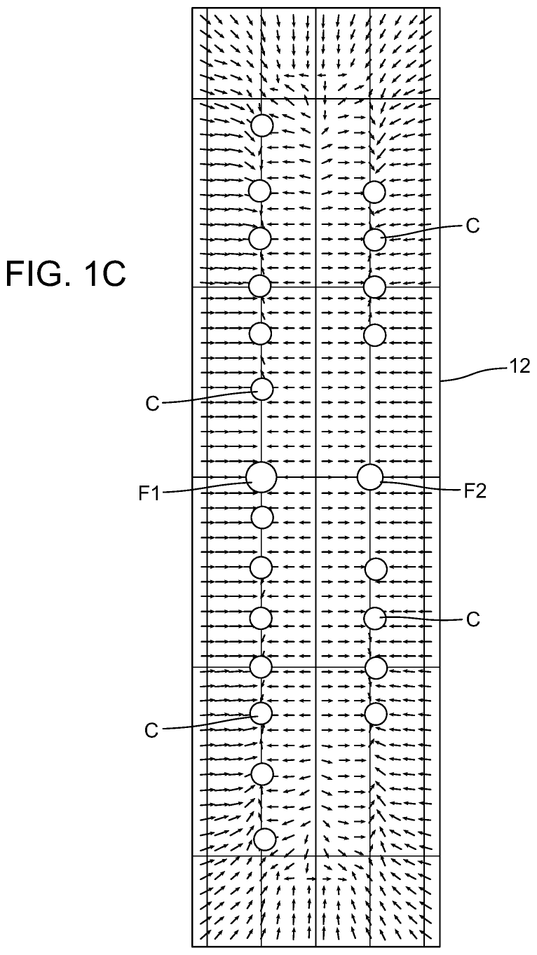

Reference is now made to FIGS. 1A-1C which illustrate a first possible embodiment of the new and improved microfluidic transfection device 10 that is adapted for enabling transfection of cells in a continuous flow-through and clog-free manner with maximal uniformity, consistency and efficiency, and minimal adverse effects. As illustrated, the microfluidic transfection device 10 includes a flow channel 12, and an electric field generator, generally designated by reference numeral 14.

More specifically, the flow channel 12 allows for passage of the target cells and the transfection agent suspended in a carrying solution. The flow channel 12 has (a) an inlet 18, (b) an inlet section 20, that may extend substantially along a first tangent, (c) an intermediate arcuate section 22, (d) an outlet section 24, that may extend substantially along a second tangent, and (e) an outlet 26 for cells transfected with the transfection agent.

The electric field generator 14 is adapted to generate an electric field across a width of the flow channel 12 at the outlet section 24 whereby the target cells continuously flowing through the flow channel are transfected with the transfection agent in an efficient and effective manner under combined physical perturbations including electrical field and hydro-pressure.

For purposes of this document, “target cells” refers to peripheral blood mononuclear cells, including T cells, B cells, lymphocytes, monocytes, natural killer cells, dendritic cells, and many cell lines including Jurkat, MC 38, and RAW 264.7 cell lines.

For purposes of this document, “transfection agent” refers to_DNA, RNA, proteins, and other gene editing molecules and conjugated nano particles.

For purposes of this document, “carrying solution” refers to any solution adapted to carry the target cells and transfection agent in suspension through an electric field. Examples include, but are not necessarily limited to various buffered solutions including saline based, phosphate based, HEPES (4-2-hydroxyethyl-1-piperazineethanesulfonic acid) based, and cell-culture-media based.

The flow channel 12 is substantially rectangular in cross section and includes a height H and a width W, where H>W. Typically, the flow channel 12 has a width W of between about 10 μm and about 250 μm and an aspect ratio of channel cross sectional area (H/W) of from about 1.5 to about 4.5. The flow channel 12 is oriented so that the larger cross sectional height H is oriented vertically and the width W lies substantially in a plane including the turns or loops of the intermediate arcuate section 22.

In the embodiment of the microfluidic transfection device 10 illustrated in FIG. 1A, the inlet section 20, which extends straight along a first tangent, has a length of between about 0.6 mm and about 30 mm. The intermediate arcuate section 22, has a spiral configuration including approximately 1 to 10 complete loops 30 of increasing radius as the suspension moves in a downstream direction through the channel. The innermost half-circle loop 32 has a radius of between about 0.5 mm and about 20 mm. The loops incrementally increase in radius by from about 2.5 W to about 5 W. The outermost half-circle loop 34 has a radius of between about 0.525 mm and about 25 mm. The overall length of the intermediate arcuate section 22 is between about 3 mm and about 350 mm. The outlet section 24, which extends substantially straight along a second tangent, has a length of between about 0.6 mm and about 30 mm.

When particles or cells C suspended in solution are passed through such a flow channel 12 having a combined configuration of spiral and straight sections, the cells will be forced to align gradually, as they move along with the fluid from the inlet 18 to the outlet 26, and form a loop-wall of cells C in the intermediate arcuate section 22 at Point A of FIG. 1A, as depicted in the cross-section view in 1B. In the straight outlet section 24 leading to the outlet 26 at Point B of FIG. 1A, these cells will be forced to align and form two vertical walls of cells C (parallel with the channel side walls at about halfway between the center and the sides of the channel) before exiting at the outlet 26. The underlying mechanism driving this cell alignment can be attributed to the combined inertial lifting, Dean, and drag forces in the spiral section (see FIG. 1B) and lift and drag forces in the straight section (see 1C). If the spiral portion of the channel 12 is sufficiently long, all cells C will be pushed to the left cell wall and continuously focused into a single position at the midpoint (see point F1 in FIG. 1B). Conversely, If the straight section 24 is sufficiently long, all cells C will be continuously pushed and focused to single positions at the midpoints of both the left and right cell walls (see points F1 and F2 in FIG. 1C).

The (a) cross sectional shape, area and orientation of the flow channel 12 and (b) the length and geometry of the inlet section 20, intermediate arcuate section 22 and outlet section 24 work together to align and focus the cells suspended in the carrying solution to enable consistent electric field exposure to all cells passing through the outlet section 24, resulting in more efficient and effective transfection with the transfection agent whether that is nanoparticles, DNA plasmids or other genetic materials.

This technology delivers clog-free transfection with throughput up to 100 million cells/min and with transfection consistency at the individual cell level. It enables cell transfection in an automatic and closed-form means to achieve the highest-possible transfection efficiency with better cell health and minimal adverse effect. It is operable with DC power and at a manageable flow rate (between about 0.5 mL/min and about 5 mL/min), and easily adoptable due to its single-inlet and single-outlet design.

The electric field generator 14, illustrated in FIG. 1A, includes a first set of electrodes 40 positioned adjacent to the outlet section 24 of the flow channel 12. More specifically the first set of electrodes 40 includes a first, positive electrode 42 on a first side of the outlet section 24 and a second, negative electrode 44 on a second, opposite side of the outlet section. The two electrodes 42, 44 are connected to a voltage source 46 that is adapted to apply a voltage potential, typically in a range from 4 to 24 Volts DC across the electrodes.

In the alternative embodiment of the microfluidic transfection device 10′ illustrated in FIG. 2, the intermediate arcuate section 22, between the inlet 18 and the outlet 26, comprises two 180-degree turns 50. The 180-degree reversing turns 50 may have a radius of curvature of between about 2 W to about 10 W. The straight sections 52 between the reversing turns 50 may have a length of between about 20 mm and about 100 mm. The overall length of the intermediate arcuate section 22 is between about 60 mm and about 300 mm.

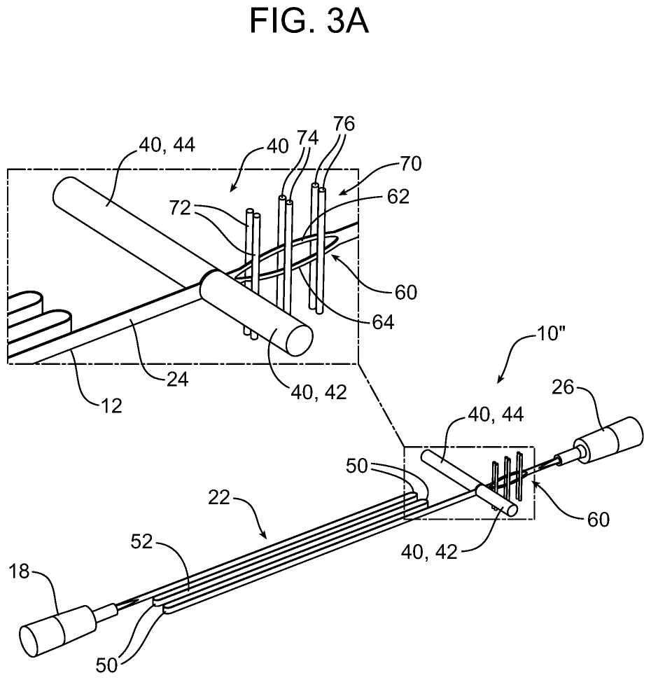

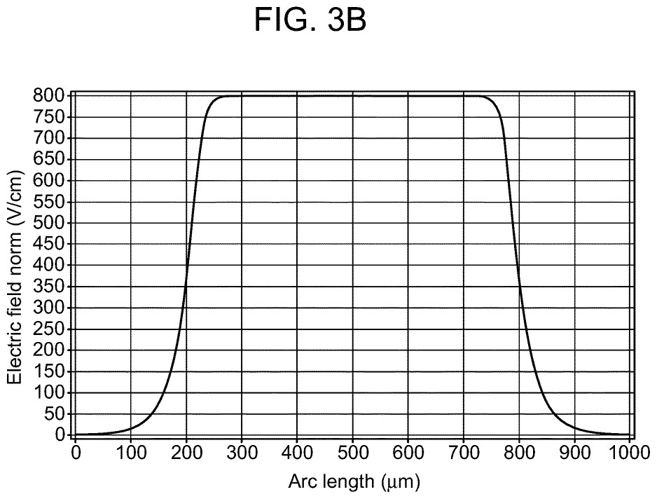

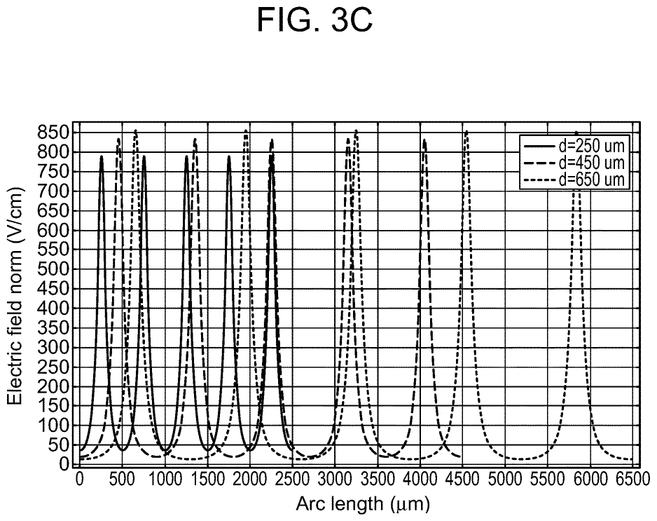

In yet another alternative embodiment illustrated in FIG. 3A, the intermediate arcuate section 22 includes four 180-degree reversing turns 50 having a radius of curvature of between, for example about 2 W to about 10 W. The straight sections 52 between the reversing turns 50 may have a length of between about 15 mm and about 75 mm. The overall length of the intermediate arcuate section may be between about 60 mm and about 300 mm.

The electric field generator 14, shown in FIG. 3A, includes a first set of electrodes 40 (note positive electrode 42 at a first side of the flow channel outlet section 24 and negative electrode 44 at a second, opposite side of the flow channel outlet section). In the illustrated embodiment, the electrodes 42, 44 have first longitudinal axes extending substantially perpendicular to the flow channel 12 and in a common plane with the outlet section of the flow channel. While most metallic materials can be used as electrodes 42, 44, metallic rods of medical grade including stainless steel (316L) and titanium alloy (Ti₆Al₄V) are better suited as cathode and platinum as anode.

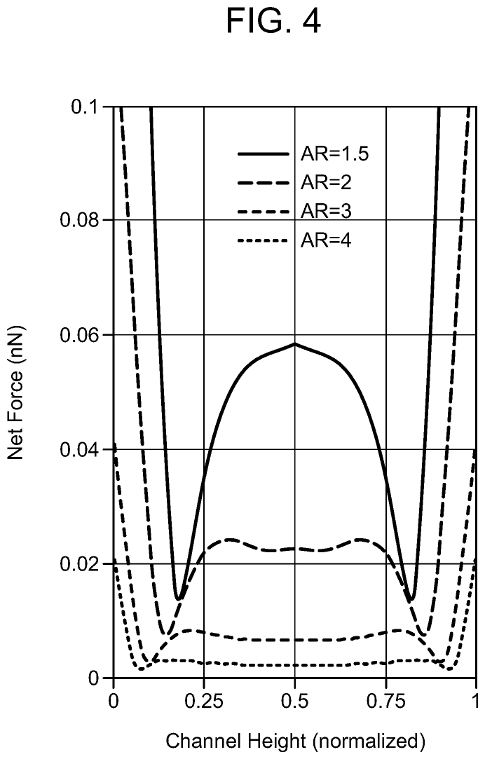

A bifurcation 60 is provided in the outlet section 24 immediately (i.e. within 0.1 to 5.0 mm) downstream from the first set of electrodes 40. The bifurcation 60 includes an upper branch 62 and a lower branch 64 of equal width and height. Thus, the bifurcation 60 is on the height or vertical axis of the flow channel 12. Each bifurcation section 62, 64 has a length of between about 1 mm and about 5 mm before they join back together again before reaching the outlet 26. The width of the two bifurcated branches is typically kept the same as the channel width W, and its height is less than the channel height H. The bifurcation 60 functions to introduce a change in hydro pressure to further perturb cell membranes as well as bring cells and transfection agents closer for enhanced delivery, and at the same time provide a second physical means, hydro-pressure, in addition to the electrical field such that the two physical perturbations can be tuned in concert to ensure better cell health (here, it should also be noted that bifurcations 60 are provided in the embodiments of FIGS. 1A and 2 to introduce a change in hydro pressure for the same beneficial results). FIG. 4 is a graph illustrating hydro-pressure forces as a function of aspect ratio of the cross section of either the flow channel 12 or the bifurcated branches 62, 64.

The electric field generator 14 also includes a second set of electrodes 70 on substantially opposite sides of the upper and lower sections 62, 64 of the bifurcation 60. The second set of electrodes 70 illustrated in FIG. 4 includes three cooperating pair of electrodes 72, 74, 76 sandwiching the flow channel 12. Each electrode 72, 74, 76 of the second set of electrodes 70 includes a second longitudinal axis that extends perpendicular to but is offset from the outlet section of the flow channel 12. Thus, in the illustrated embodiment, the longitudinal axes of the first set of electrodes 40 extends in a substantially horizontal plane while the longitudinal axes of the (thinner-in-cross-section) second set of electrodes 70 extend in a substantially vertical plane. Note also how the bifurcation 60 of outlet section 24 is received between the electrode pairs 72, 74, 76.

The second set of electrodes 70 provide additional shorter electric-field exposure to cells C while they are in the channel 24 or in the bifurcated sections 62, 64 to further enhance insertion of genetic materials into cells and cell nuclei. They will not need to be connected to any electric power in the case where such additional electric-field exposure is deemed unnecessary.

In these microfluidic transfection devices 10, 10′, 10″, the time duration in which cells C travel through the electric field and the bifurcated section will be used to control the physical perturbation time. In this way, flow velocity and the span of the electric field and length of the bifurcation section 60 become parameters for controlling exposure time. While the flow velocity can be determined from the flow rate set at the inlet 18, the electric-field span is set by utilizing electrodes 42, 44, 72, 74, 76 of different diameters and spacing. The change of hydro-pressure is achieved by having a narrower height, typically from about 0.375 H to 0.75 H, for the bifurcated branches 62, 64.

For example, with a nominal flow rate of from 0.8 to 1.2 mL/min, the diameter of the horizontal electrodes 42, 44 are to be selected to provide longer-time electric-field exposure by cells C (e.g., in a range from 100s μs to a few ms with the use of electrodes having diameters ranging from 100s μm to a few mm), and the selection for the diameter of the vertical electrodes 72, 74, 76 is expected to provide shorter-time exposure by cells (e.g., in a range from a few 100s ns to 100s us with the use of electrodes having diameters in the 100s μm or smaller). A sample electric field generated by a pair of horizontal electrodes 42, 44 (~500 μm in diameter) is given in FIG. 3B, and three samples of the resulting electric field generated by 5-pair of vertical round-rod electrodes (with diameters from 250 um to 650 um) placed in a row are shown in FIG. 3C. These pulse-like electric fields can be tuned by utilizing electrodes 72, 74, 76 of different diameters and placing them at different positions. One or more of the different sets of electrodes 40, 72, 74, 76 could also be connected to different voltage sources, providing different applied voltages. Minor adjustment of flow rate will provide additional fine tuning to the electric-field exposure time, but any large upward adjustment of flow rate may demand a higher pump pressure for operations.

It should be noted that all cells C, while moving along with the flow, are moving forward in a spinning manner due to the presence of a flow-velocity gradient from the center to the sidewalls of the channel 12. When cells C move through the electric field adjacent the electrodes 40, 72, 74, 76 in this manner, all cells will have their membranes aligned perpendicular to the electric field. This is crucial for facilitating maximal and uniform electric-field exposure by all cells C. It is also worth emphasizing that while forcing cells C to align into single focused position(s) will possibly deliver even higher, or the highest, uniformity and consistency in electric field exposure, doing so will require the device 12 to have a sufficiently long channel 12, which in turn will demand a higher pump pressure for operations. For low-pressure operations, microfluidic transfection devices 10, 10′, 10″ can be designed to just take advantage of the vertical-wall alignment of cells C. Should single-line particle alignment become desirable, such as in devices with combined cell sorting-and-separation capabilities and cell-transfection capability, similar microfluidic transfection devices can be designed and fabricated by adding more loops or turns to increase the overall length of the fluidic channel 12.

Any of the above embodiments of the microfluidic transfection device 10, 10′, 10″ illustrated in FIG. 1A, 2 or 3A is useful in a method for transfecting target cells with a transfection agent in a suspension. That method includes the steps of: (1) introducing the suspension into an inlet 18 of a flow channel 12, (2) flowing the suspension through the flow channel from the inlet serially through (a) an inlet section 20, that may extend substantially along a first tangent, (b) an intermediate arcuate section 22, (c) an outlet section 24 including a vertically bifurcated section 60, that may extend substantially along a second tangent, and (d) an outlet 26 of the flow channel, and (3) applying an electric field across a width of the flow channel adjacent the bifurcation 60 whereby the target cells continuously flowing through the flow channel are transfected with the transfection agent under combined physical perturbations including electrical field and hydro-pressure.

The step of applying the electric field may also include applying a voltage potential across a first set of electrodes 40 on substantially opposite sides of the outlet section 24 of the flow channel 12 (See FIG. 1A). In addition, the method includes passing the suspension with the target cells and the transfection agent through the bifurcation 60 in the outlet section 24 of the flow channel 12 downstream from the first set of electrodes 40.

Still further, the method also includes applying a voltage potential across a second set of electrodes 70 on substantially opposite sides of the bifurcation 60 in the outlet section 24 of the flow channel 12.

In any of the many possible embodiments, the method may include passing the suspension with the target cells and the transfection agent through the flow channel 12 of rectangular cross section having a height H and a width W, where H>W, and wherein turns of the intermediate arcuate section and the width W share a common plane where the height H is generally oriented in a vertical direction while the width W is generally oriented in a horizontal direction. Still further, in at least some embodiments, the method includes including a spiral having about 1 to about 5 complete loops in the intermediate arcuate section 22 as shown in FIG. 1A. In still other embodiments, the method includes including at least two turns in the intermediate arcuate section as shown in FIGS. 2 and 3A.

Advantageously, the microfluidic transfection device 10 and method take advantage of the inertial focusing mechanism of the microfluidic device 12 to align and focus cells C prior to exposure to electrical field or hydro-pressure change. Specifically, the microfluidic cell transfection technology embodied in the device 10 and method take advantage of the microfluidic device 12 having uniquely designed channel layout and cross-section. When cells C in suspension are fed through such a 3D microfluidic device 10, 10′, 10″, the cells experience forces that will drive them to form vertical alignment patterns and move forward in a spinning manner. These attributes will not only ensure individual cells C to be exposed consistently to the electric field and hydro pressure in an equal-strength and -duration manner, but also provide hydro-pressure “massaging” to these cells to maintain a high level of cell health and viability. Moreover, unlike other flow-through systems, this technology utilizes a combined electro-hydroporation method to gently induce transmembrane pores and provide tuning of the electric-field strength and hydro-pressure in concert to achieve the highest possible transfection efficiency with minimal unwanted genetic changes.

With the 3D microfluidic transfection technology described in this document, cells can be transfected in a continuous flow-through and clog-free manner. Prior to electric-field exposure, all cells, while moving along with the flow, will be forced to take a fixed path through the electric field and have their membranes aligned perpendicular to the electric-field. In this way, all cells will be exposed to the electric field uniformly in a maximal and controllable amount.

### EXPERIMENTAL

Prototypes of 3D microfluidic transfection devices 10, 10′ and 10″ have been fabricated using a cost-effective 3D printing method along with commercially available UV-curing polymer resins as well as a biocompatible poly-ethylene glycol acrylate (in either PEGDA, PEGTA, or PEGTA format) based UV-curing polymer.

Testing of these fabricated microfluidic transfection devices has been conducted through cell transfection experiments. In these experiments, a mouse colon carcinoma MC38 cell line (Kerafast) was used and magnetic nanoparticles (MNPs) were used as transfection materials. MNPs were synthesized by coating 15 nm iron oxide nanocrystals with a phospholipid-poly(ethylene glycol) copolymer through a dual solvent exchange method. After coating, MNPs were conjugated with a positively charged peptide and labeled with Dil. The hydrodynamic size of MNPs is approximately 30 nm as measured by dynamic light scattering. Before transfection experiments, MC38 cells were detached with trypsin-EDTA and washed with PBS. After washes, the cells were resuspended at a concentration of 1×10⁶ cells/mL in PBS. MNPs were added to the cell suspension at a concentration of 8 μg Fe/mL. The mixture of MNPs and cells were added to a 1 ml syringe connected to the microfluidic channel through silicon tubing. The injection speed was controlled by using a syringe pump, which in turn set the flow rate for transfection experiments at a preselected nominal rate of 0.8 mL/min.

For these experiments, two microfluidic devices with two 180-degree turns and a pair of horizontal electrodes (1 mm in diameter) were used. The electrodes were connected to a DC power supply set at 8 V. In each experiment run, the power source was kept off during control-group runs and turned on during the testing group runs. The flow-through cells were collected in a microplate. The collected cells were added to 1.5 mL tubes and centrifuged at 200 g for 5 minutes. After the supernatant was removed, the cell pellets were dispersed with PBS. The fluorescence signal of the cells was analyzed with flow cytometry. In control group, no discernable (<0.2%) cell transfection activities were observed as expected. In testing group, 92.6% transfection rate was obtained for Device 1 and 82.7% for Device 2. Note that the observed difference can be attributed to the difference in the gap distance between the ends of the two horizontal electrodes because during device assembly—the gap in Device 2 was slightly larger than that in Device 1.

Testing of fabricated microfluidic transfection devices has also been conducted using the mouse macrophage cell-line RAW 264.7 cells (ATCC). In these experiments, three additional microfluidic devices in two 180-degree turns with a pair of horizontal electrodes (1 mm in diameter) along with a bifurcation segment were used. With all other experimental conditions being the same as for the MC38 cells, in control group no discernable (<0.5%) cell transfection was observed, and in testing group almost 100% transfection efficiency, 96.9% for Device 3, 98.7% for Device 4 and 100% for Device 5, was observed. Achieving these high efficiency values indicates the superior performance of this continuous and high-throughput 3D microfluidic transfection device.

Each of the following terms written in singular grammatical form: “a”, “an”, and “the”, as used herein, means “at least one”, or “one or more”. Use of the phrase “One or more” herein does not alter this intended meaning of “a”, “an”, or “the”. Accordingly, the terms “a”, “an”, and “the”, as used herein, may also refer to, and encompass, a plurality of the stated entity or object, unless otherwise specifically defined or stated herein, or, unless the context clearly dictates otherwise. For example, the phrase: “a transfection agent”, as used herein, may also refer to, and encompass, a plurality of transfection agents.

Each of the following terms: “includes”, “including”, “has”, “having”, “comprises”, and “comprising”, and, their linguistic/grammatical variants, derivatives, or/and conjugates, as used herein, means “including, but not limited to”, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof.

The phrase “consisting of”, as used herein, is closed-ended and excludes any element, step, or ingredient not specifically mentioned. The phrase “consisting essentially of”, as used herein, is a semi-closed term indicating that an item is limited to the components specified and those that do not materially affect the basic and novel characteristic(s) of what is specified. Terms of approximation, such as the terms about, substantially, approximately, etc., as used herein, refers to ±10% of the stated numerical value.

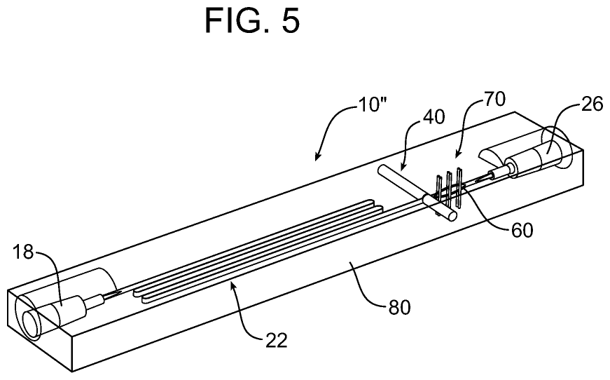

Although the microfluidic transfection device 10 and method of this disclosure have been illustratively described and presented by way of specific exemplary embodiments, and examples thereof, it is evident that many alternatives, modifications, or/and variations, thereof, will be apparent to those skilled in the art. For example, as shown in FIG. 5, the microfluidic transfection device 10″ of FIG. 3A may be held in a housing 80. As another example, the intermediate arcuate section 22 may include any number of turns such as 5 or more turns of 180 degrees. In other embodiments, the turns may be of less or more than 180 degrees. Still further, the first set of electrodes 40 may include more than one pair 42, 44, and the second set of electrodes may be placed (a) upstream, (b) downstream, or (c) upstream and downstream from the first set of electrodes 40. Accordingly, it is intended that all such alternatives, modifications, or/and variations, fall within the spirit of, and are encompassed by, the broad scope of the appended claims.

What is claimed:

1. A 3D microfluidic transfection device adapted for transfecting target cells with a transfection agent, comprising:
a flow channel for the target cells and the transfection agent suspended in a carrying solution, the flow channel having (a) an inlet, (b) a bifurcation, and (c) an outlet; and
an electric field generator adapted to generate an electric field across a width of the flow channel whereby the target cells continuously flowing through the flow channel are transfected with the transfection agent under combined physical perturbations including electrical field and hydro-pressure.

2. The microfluidic transfection device of claim 1, wherein the electric field generator includes (a) electrodes positioned adjacent to the flow channel and (b) a voltage source connected to the electrodes and adapted for applying a voltage potential across the electrodes.

3. The microfluidic transfection device of claim 2, wherein the electrodes include a first set of electrodes on substantially opposite sides of the flow channel adjacent the bifurcation.

4. The microfluidic transfection device of claim 3, wherein the bifurcation is downstream from the first set of electrodes.

5. The microfluidic transfection device of claim 4, wherein the electric field generator further includes a second set of electrodes on substantially opposite sides of the bifurcation.

6. The microfluidic transfection device of claim 5, wherein the first set of electrodes include first longitudinal axes substantially perpendicular to the flow channel and in a common plane with the flow channel and the second set of electrodes include second longitudinal axes perpendicular to and offset from the flow channel at the bifurcation.

7. The microfluidic transfection device of claim 4, wherein the bifurcation merges back again into a single flow channel downstream from the second set of electrodes and upstream from the outlet.

8. The microfluidic transfection device of claim 1, wherein (a) the flow channel has a rectangular cross section having a height H and a width W, where H>W, and (b) turns of the intermediate arcuate section and the width W share a common plane.

9. The microfluidic transfection device of claim 8, wherein the flow channel includes at least one turn.

10. The microfluidic transfection device of claim 9, wherein the flow channel has a width of between about 10 μm and about 250 μm and an aspect ratio of channel cross sectional area (H/W) of from about 1.5 to about 4.5.

11. The microfluidic transfection device of claim 8, wherein the flow channel includes an intermediate arcuate section generally in a form of a spiral including about 1 to about 10 loops.

12. The microfluidic transfection device of claim 11, wherein the flow channel includes a plurality of loops wherein an innermost loop has a radius of between about 0.5 mm and about 20 mm and the outermost loop has a radius of between about 0.525 mm and about 25 mm.

13. A microfluidic transfection device, comprising:
a microfluidic device including a flow channel for the target cells and the transfection agent suspended in a carrying solution, the flow channel having (a) an inlet, (b) an intermediate arcuate section adapted to advantageously position target cells within the flow channel for transfection, (c) an outlet section including a bifurcation, and (d) an outlet; and
an electric field generator adapted to generate an electric field across a width of the flow channel at the bifurcation of the outlet section whereby the target cells continuously flowing through the flow channel are transfected with the transfection agent under combined physical perturbations including electrical field and hydro-pressure.

14. A method of transfecting target cells with a transfection agent in a suspension, comprising:
introducing the suspension into an inlet of a flow channel;
flowing the suspension through the flow channel from the inlet through a bifurcation to an outlet; and
applying an electric field across a width of the flow channel adjacent the bifurcation whereby the target cells continuously flowing through the flow channel are transfected with the transfection agent.

15. The method of claim 14, wherein the applying of the electric field includes applying a voltage potential across a first set of electrodes on substantially opposite sides of the flow channel.

16. The method of claim 15, further including passing the suspension with the target cells and the transfection agent through the bifurcation downstream from the first set of electrodes.

17. The method of claim 16, wherein the applying of the electric field further includes applying a voltage potential across a second set of electrodes on substantially opposite sides of the bifurcation in the flow channel.

18. The method of claim 17, including passing the suspension with the target cells and the transfection agent through the flow channel of rectangular cross section having a width W and a height H, where H>W, and wherein any turns in the flow channel and the width W share a common plane.

19. The method of claim 18, including at least one turn in an intermediate arcuate section of the flow channel.

20. The method of claim 18, including a spiral having about 1 to about 10 complete loops in an intermediate arcuate section of the flow channel.
